(19) United States
(12) Patent Application Publication
(10) Pub. No.: US 2007/0020205 A1
(43) Pub. Date: Jan. 25, 2007

(54) Two-coat cosmetic product, uses thereof and makeup kit containing this product

(75) Inventors: Xavier Blin, Paris (FR); Shaoxiang Lu, Plainsboro, NJ (US); Hy Si Bui, Piscataway, NJ (US)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/485,283

(22) Filed: Jul. 13, 2006

Related U.S. Application Data
(60) Provisional application No. 60/698,791, filed on Jul. 13, 2005.

(51) Int. Cl. A61K 8/81; A61K 8/97

(52) U.S. Cl. 424/61; 424/70.7; 424/70.11; 424/74

(57) ABSTRACT

Product containing a first composition and a second composition, the first composition containing at least one resin chosen from rosin and derivatives thereof, and hydrocarbon-based resins, and mixtures thereof, the resin having a number-average molecular mass of less than or equal to 10 000 g/mol, and the second composition, which is different from the first, containing at least one non-volatile fatty substance. The invention also relates to a makeup process and also to a makeup kit containing the product. This product is in particular a lipstick, a mascara, a foundation or a nail varnish.

### REFERENCE TO PRIOR APPLICATIONS

This application claims priority to U.S. provisional application 60/698,791 filed Jul. 13, 2005, incorporated herein by reference.

### FIELD OF THE INVENTION

The present invention relates to a product comprising at least two compositions that may be applied successively to the skin either of the face or of the body, to the lower and upper eyelids, to the lips and to the integuments, for instance the nails, the eyebrows, the eyelashes, or the hair. The present invention also relates to a process for making up the face and the body using these two compositions.

### DETAILED DESCRIPTION OF THE PREFERRED EMBODIMENTS

Each product or composition according to the invention may be a foundation, a makeup rouge, an eyeshadow, a concealer product, a blusher, a loose or compacted powder, a lipstick, a lip balm, a lip gloss, a lip pencil, an eye pencil, a mascara, an eyeliner, a nail varnish, a body makeup product or a skin colouring product.

One object of the present invention is to provide a novel route for formulating a cosmetic product, in particular a makeup product, of the type comprising two compositions to be applied successively one after the other. This cosmetic product may advantageously have good gloss and/or staying power properties. In addition the invention product has excellent hold, transfer-resistance and migration-resistance properties, which, when allied with the glossy and non-greasy appearance, make it a product that is particularly suitable for producing lip makeup products such as lipsticks and lip glosses, eye makeup products such as mascaras, eyeliners and eyeshadows, and nail or hair makeup products.

One subject of the present invention is thus a cosmetic product intended to be applied to keratin materials, comprising a first composition and a second composition, the first composition comprising at least one resin chosen from rosins, rosin derivatives and hydrocarbon-based resins, and mixtures thereof, the said resin having a number-average molecular mass of less than or equal to 10 000 g/mol, and the second composition, which is different from the first, comprising at least one high-viscosity oil.

The product of the invention is in particular a makeup product for the skin, the nails or the hair.

The term “makeup product” means a product containing a colouring agent allowing the deposition of a colour onto a keratin material (the skin or the integuments) of a human being by applying to the keratin material products such as lipsticks, makeup rouges, eyeliners, foundations, self-tanning products or semi-permanent makeup products (tattoos).

The The product according to the invention preferably comprises at least two cosmetically acceptable compositions packaged separately or together in the same packaging article or in at least two separate packaging articles.

Preferably, these compositions are packaged separately and advantageously in separate packaging articles.

The subject of the present invention is thus, in particular, a cosmetic makeup product in the form of a lipstick, a foundation, a mascara, a makeup rouge, an eyeliner, a lipstick, a nail varnish, a product especially having care properties, a mascara, an eyeliner, a concealer product or a makeup. product for the body (such as a tattoo) or for the hair.

A subject of the invention is also a makeup kit containing a cosmetic makeup product as defined above, in which the various compositions are packaged separately and are advantageously accompanied by one or more suitable applicators. These applicators may be fine brushes, coarse brushes, pens, pencils, felts, feathers, sponges, tubes and/or foam tips.

The first composition of the The product according to the invention may constitute a basecoat applied onto the keratin material, and the second composition a topcoat. However, it is possible to apply under the first coat an undercoat that may or may not have the constitution of the second coat.

It is also possible to deposit an overcoat onto the second coat, which may have a constitution identical to or different from that of the first coat. Preferably, the makeup obtained is a two-coat makeup.

The second composition may also constitute a basecoat applied to the keratin material and the first composition a topcoat.

In particular, the basecoat is preferably a lipstick, a foundation, a mascara, a lip gloss, an eyeliner, a nail varnish, a nailcare product or a body makeup product, and the topcoat is a care or protective product.

The invention also relates to a process for making up the skin and/or the lips and/or the integuments, which comprises applying a cosmetic product as defined above to the skin and/or the lips and/or the integuments.

A subject of the invention is also a cosmetic process for caring for or making up human skin and/or lips and/or integuments, which comprises applying to the skin, the lips and/or the integuments a first coat of a first composition comprising at least one resin chosen from rosins, rosin derivatives and hydrocarbon-based resins, and mixtures thereof, the said resin having a number-average molecular mass of less than or equal to 10 000 g/mol, and then in applying, to all or part of the first coat, a second coat of a second composition comprising at least one high-viscosity oil.

The The product according to the invention may be applied to the skin of either the face or the scalp and of the body, the lips, the inner edge of the lower eyelids, and the integuments, for instance the nails, the eyelashes, the hair, the eyebrows, or even body hair. The second composition may form patterns and may be applied with a pen, pencil or any other instrument (sponge, finger, fine brush, coarse brush or feather). This makeup may also be applied onto makeup accessories, for instance false nails, false eyelashes or wigs, or alternatively, onto dots or patches that adhere to the skin or the lips (such as beauty spots).

Another subject of the invention is the use of a cosmetic product comprising a first composition and a second composition, the first composition comprising at least one resin chosen from rosins, rosin derivatives and hydrocarbon-based resins, and mixtures thereof, the said resin having a number-average molecular mass of less than or equal to 10 000 g/mol, and the second composition comprising a high-viscosity oil, to give the skin and/or the lips and/or the integuments a cosmetic result that is comfortable and/or glossy and/or transfer-resistant and/or migration-resistant and/or that has good hold.

First Composition

The first composition according to the invention comprises at least one resin chosen from rosins, rosin derivatives and hydrocarbon-based resins, and mixtures thereof, the said resin having a number-average molecular mass of less than or equal to 10 000 g/mol, including 9,000, 8000, 7000, 6000, 5000, 4000, 3000, 2500, 2000, 1000, etc. g/mol and all values and subranges therebetween.

The first composition advantageously has a resistance to oil of greater than or equal to 50%.

The said composition is advantageously capable of forming a film that has a deposit whose resistance to oil is greater than or equal to 50%, for example 55%, 60% or even 65%.

The resistance to oil may be measured according to the following method.

Three glass slides are prepared, each covered with a sheet of collagen as follows.

A sheet of collagen (naturin artificial gut, thickness 0.06 mm, a. 0.10 mm, d. 120 mm) with a side length of 5 cm by 10 cm is prepared at a temperature of 28° C., and is conditioned for at least two hours at 90% relative humidity (RH).

The sheet of collagen is returned to the open air and is immediately attached securely and entirely to a 4.6 cm by 7.6 cm glass slide. The sheet of collagen is attached to the reverse of the glass slide with 3M Scotch tape. The surface of the collagen should be flat and free of folds. Each slide is left under ambient conditions for 24 hours before performing the test.

A 5 cm by 10 cm rectangle is cut out of a Styrofoam plate (Amoco Selectables Plastics DL Tableware type) using a knife and a ruler by following the contour of a glass slide. The The product according to the invention is applied to each glass slide and to the Styrofoam rectangle as follows. The composition is applied using a 25 μm mechanical applicator (bar coater).

The plates are left under ambient conditions for 24 hours.

Three drops of olive oil (about 0.075 g) spread out with a brush are applied to each of the three slides covered with collagen and with composition. The excess is dried up using a Kimwipes paper towel and the slides are left for 30 minutes at room temperature. Three white Styrofoam discs 4 cm in diameter are cut out.

The white Styrofoam disc is securely attached with double-sided tape to the end of a 2 kg mass, and, by exerting a pressure of 175 g/cm², the weight is gently placed on the surface of a plate (product side) and the weight is rotated one and a half times about itself over 3 to 5 seconds, while maintaining the initial pressure. The weight is raised and the Styrofoam disc is recovered. The measurement is performed for each glass slide with a clean Styrofoam disc.

The percentage of reflectance:
of the deposit of product applied to the rectangular Styrofoam sample (reference A),
of the clean white Styrofoam disc (reference B),
of the disc detached from the weight after the pressure has been applied to the slide coated with cosmetic product (reference C)
   is then measured. The reflectance is measured over a wavelength range of between 400 and 700 nm using a spectral analyser (aperture 25 mm in diameter) with a D65/10° illuminant. The wavelength of the reflectance minimum is chosen for the “stained” disc. At this wavelength, the resistance is calculated according to the equation
   100*(1−[(C-B)/(A-B)])

The resistance to oil is equal to the mean of the three measurements.

Resin

The resin used in the composition according to the invention preferably has a number-average molecular mass of less than or equal to 10 000 g/mol, especially ranging from 250 to 10 000 g/mol, preferably less than or equal to 5000 g/mol, especially ranging from 250 to 5000 g/mol, better still less than or equal to 2000 g/mol, especially ranging from 250 to 2000 g/mol and better still less than or equal to 1000 g/mol, especially ranging from 250 to 1000 g/mol.

The number-average molar masses (Mn) are determined by gel permeation liquid chromatography (THF solvent, calibration curve established with linear polystyrene standards, refractometric detector).

The resin of the composition according to the invention is advantageously a tackifying resin. Such resins are described especially in the Handbook of Pressure Sensitive Adhesive, edited by Donatas Satas, 3rd edition, 1989, pp. 609-619.

The resin of the composition according to the invention may be chosen from rosin or a derivative thereof and hydrocarbon-based resins, and mixtures thereof.

The term “hydrocarbon-based” means a compound predominantly comprising carbon and hydrogen, and possibly heteroatoms such as oxygen, nitrogen or sulfur. The hydrocarbon-based compound preferably consists of carbon and hydrogen.

The rosin is a mixture predominantly comprising organic acids known as rosin acids (mainly acids of abietic type and of pimaric type) . Three types of rosin exist: the rosin (gum rosin) obtained by incision on live trees, wood rosin, which is extracted from pine wood or stumps, and tall oil (tall oil rosin), which is obtained from a by-product originating from the production of paper.

The rosin derivatives may be derived in particular from the polymerization, hydrogenation and/or esterification (for example with polyhydric alcohols such as ethylene glycol, glycerol or pentaerythritol) of rosin acids. Examples that may be mentioned include the rosin esters sold under the reference Foral 85, Pentalyn H and Staybelite Ester 10 by the company Hercules; Sylvatac 95 and Zonester 85 by the company Arizona Chemical, or Unirez 3013 by the company Union Camp.

The hydrocarbon-based resins may be chosen from olefinic polymers of low molecular mass, which may be classified, according to the type of monomer they comprise, as indene polymers, pentadiene resins, cyclopentadiene dimer resins and terpenic resins.

The indene polymers may be chosen from polymers derived from the polymerization in major proportion of indene monomer and in minor proportion of monomers chosen from styrene, methylindene and methylstyrene, and mixtures thereof. These polymers may optionally be hydrogenated, and may have a molecular weight ranging from 200 to 1500 g/mol.

The indene hydrocarbon-based polymer is preferably a block copolymer obtained from indene and from styrene or a styrene derivative.

According to one preferred embodiment, the resin is chosen from indene resins, in particular the hydrogenated indene/methylstyrene/ styrene copolymers sold under the name “Regalite” by the company Eastman Chemical, such as Regalite R 1100, Regalite R 1090, Regalite R-7100, Regalite R 1010 Hydrocarbon Resin and Regalite R 1125 Hydrocarbon Resin.

Examples of indene polymers that may be mentioned include those sold under the reference Escorez 7105 by the company Exxon Chem., Nevchem 100 and Nevex 100 by the company Neville Chem., Norsolene S105 by the company Sartomer, Picco 6100 by the company Hercules and Resinall by the company Resinall Corp.

Other examples of indene polymers that may be mentioned include pentadiene and indene resins, which are derived from the polymerization of a mixture of pentadiene and indene monomers such as those described above, for instance the resins sold under the name Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear.

The pentadiene resins may be chosen from those derived from the majority polymerization of the 1,3-pentadiene (trans or cis-piperylene) monomer and of minor monomers chosen from isoprene, butene, 2-methyl-2-butene, pentene and 1,4-pentadiene, and mixtures thereof. These resins may have a molecular weight ranging from 1000 to 2500 g/mol.

Such 1,3-pentadiene resins are sold, for example, under the references Piccotac 95 by the company Eastman Chemical, Escorez 1304 by the company Exxon Chemicals, Nevtac 100 by the company Neville Chem. or Wingtack 95 by the company Goodyear.

The cyclopentadiene dimer resins may be chosen from those derived from the polymerization of first monomers chosen from indene and styrene, and of second monomers chosen from cyclopentadiene dimers such as dicyclopentadiene, methyldicyclopentadiene and other pentadiene dimers, and mixtures thereof. These resins generally have a molecular weight ranging from 500 to 800 g/mol, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.

The terpenic resins may be chosen from those derived from the polymerization of at least one monomer chosen from α-pinene, β-pinene and limonene, and mixtures thereof. These resins may have a molecular weight ranging from 300 to 2000 g/mol. Such resins are sold, for example, under the names Piccolyte A115 and S125 by the company Hercules, and Zonarez 7100 or Zonatac 105 Lite by the company Arizona Chem.

Hydrocarbon-based resins that may also be mentioned include certain resins sold under the name Eastotac C6-C20 Polyolefin by the company Eastman Chemical Co., under the reference Escorez 5300 by the company Exxon Chemicals, or the resins Nevillac Hard or Nevroz sold by the company Neville Chem., the resins Piccofyn A-100, Piccotex 100 or Piccovar AP25 sold by the company Hercules or the resin SP-553 sold by the company Schenectady Chemical Co.

One of the subjects of the present invention is a cosmetic lip makeup composition comprising at least one indene hydrocarbon-based polymer with a number-average molecular mass of less than or equal to 10 000 g/mol. This composition is advantageously capable of forming a film with a resistance to oil of greater than or equal to 50%. In this embodiment, the indene polymer may be one of the indene polymers described previously. The indene hydrocarbon-based polymer is preferably a block copolymer obtained from indene and from styrene or a styrene derivative.

The resins may be present in the composition according to the invention in any amount, particularly a content ranging from 0.1% to 40% by weight, preferably from 0.5% to 30% by weight and better still from 1% to 20% by weight, for example from 2% to 15% by weight, relative to the total weight of the composition.

The resin may have at least one glass transition temperature preferably of greater than or equal to 20° C., preferably greater than or equal to 30° C., and preferably of about 40° C. The glass transition temperature is advantageously between 20°0 C. and 300° C., for example between 30° C. and 100° C.

The glass transition temperatures indicated in the present patent application may be theoretical Tg values determined from the theoretical Tg values of the constituent monomers of each of the blocks, which may be found in a reference manual such as the Polymer Handbook, 3rd edition, 1989, John Wiley, according to the following relationship, known as Fox's law:

$$1 / Tg = \frac{\sum }{i} ( {\overline{ω}}_{i} / {Tg}_{i} ) ,$$

ω̅ᵢ being the mass fraction of the monomer i in the block under consideration and Tgᵢ being the glass transition temperature of the homopolymer of the monomer i.

The glass transition temperature (Tg) may be measured according to standard ASTM D3418-97, via differential thermal analysis (DSC, Differential Scanning Calorimetry) on a calorimeter, over a temperature range of between −100° C. and +150° C. at a heating rate of 10° C./minute in 150 μl aluminium crucibles.

Liquid Fatty Phase

The composition according to the invention may comprise a fatty phase that is liquid at room temperature (25° C.) and atmospheric pressure (760 mmHg), composed of one or more mutually compatible non-aqueous fatty substances that are liquid at room temperature, also known as organic solvents or oils.

The oil may be chosen from volatile oils and/or non-volatile oils, and mixtures thereof.

For the purposes of the invention, the term “volatile oil” means an oil that is capable of evaporating on contact with the skin or the keratin fibre in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg). The term “non-volatile oil” means an oil that remains on the skin or the keratin fibre at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C8-C16 alkanes, for instance C8-C16 isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C8-C16 esters and isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from volatile hydrocarbon-based oils containing from 8 to 16 carbon atoms, and mixtures thereof.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethyl-cyclopentasiloxane, dodecamethylcyclohexasiloxane, heptamethylhexyltrisiloxane, heptamethyloctyltri-siloxane, hexamethyldisiloxane, octamethyltrisiloxane, decamethyl tetrasiloxane and dodecamethylpentasiloxane, and mixtures thereof.

Mention may also be made of the linear volatile alkyltrisiloxane oils of general formula (I): 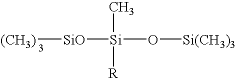
in which R represents an alkyl group containing from 2 to 4 carbon atoms and of which one or more hydrogen atoms may be substituted with one or more fluorine or chlorine atoms.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluorinated solvents such as nonafluoro-methoxybutane or perfluoromethylcyclopentane may also be used.

The composition according to the invention may also comprise at least one non-volatile oil, chosen in particular from non-volatile hydrocarbon-based oils and/or silicone oils and/or fluoro oils.

Non-volatile hydrocarbon-based oils that may especially be mentioned include:
hydrocarbon-based oils of plant origin, such as triesters of fatty acids and of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or caprylic/capric acid triglycerides, for instance those sold by the company Stearineries Dubois or those sold under the names Miglyol 810, 812 and 818 by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as parleam, and squalane, and mixtures thereof,
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpenta-decanol,
higher fatty acids such as oleic acid, linoleic acid or linolenic acid,
and mixtures thereof.

According to one embodiment of the present invention, the composition contains a polar oil, for example an alcohol chosen from fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

The non-volatile silicone oils that may be used in the composition according to the invention include non-volatile polydimethylsiloxanes (PDMS), polydimethyl-siloxanes comprising alkyl or alkoxy groups, which are pendent and/or at the end of a silicone chain, these groups each contain from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyl-trisiloxanes and 2-phenylethyltrimethylsiloxysilicates.

The fluoro oils that may be used in the invention are especially fluorosilicone oils, fluoro polyethers and fluorosilicones as described in document EP-A-847 752.

According to one embodiment, the fatty phase contains an ester oil. This ester oil may be chosen from the esters of monocarboxylic acids with monoalcohols and polyalcohols.

Advantageously, the said ester corresponds to formula (I) below:
R₁—CO—O—R₂ (I)
where R₁ represents a linear or branched alkyl radical of 1 to 40 carbon atoms and preferably of 7 to 19 carbon atoms, optionally comprising one or more ethylenic double bonds, and optionally substituted,
R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

The term “optionally substituted” means that R₁ and/or R₂ can bear one or more substituents chosen, for example, from groups comprising one or more hetero atoms chosen from O, N and S, such as amino, amine, alkoxy and hydroxyl.

Preferably, the total number of carbon atoms of R₁+R₂ is ≧9.

R₁ may represent the residue of a linear or, preferably, branched fatty acid, preferably a higher fatty acid, containing from 1 to 40 and even better from 7 to 19 carbon atoms, and R₂ may represent a linear or, preferably, branched hydrocarbon-based chain containing from 1 to 40, preferably from 3 to 30 and even better from 3 to 20 carbon atoms. Once again, preferably the number of carbon atoms of R₁+R₂ ≧9.

Examples of groups R₁ are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters include purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols, for example of fatty alcohols.

Advantageously, the esters are chosen from the compounds of formula (I) above, in which R₁ represents an unsubstituted linear or branched alkyl group of 1 to 40 carbon atoms and preferably of 7 to 19 carbon atoms, optionally comprising one or more ethylenic double bonds, and R₂ represents an unsubstituted linear or branched alkyl group of 1 to 40 carbon atoms, preferably of 3 to 30 carbon atoms and even better of 3 to 20 carbon atoms, optionally comprising one or more ethylenic double bonds.

Preferably, R₁ is an unsubstituted branched alkyl group of 4 to 14 carbon atoms and preferably of 8 to 10 carbon atoms, and R₂ is an unsubstituted branched alkyl group of 5 to 15 carbon atoms and preferably of 9 to 11 carbon atoms. Preferably, in formula (I), R₁—CO— and R₂ have the same number of carbon atoms and are derived from the same radical, preferably an unsubstituted branched alkyl, for example isononyl, i.e. the ester oil molecule is advantageously symmetrical.

The ester oil will preferably be chosen from the following compounds:
isononyl isononanoate,
cetostearyl octanoate,
isopropyl myristate,
2-ethylhexyl palmitate,
2-octyldodecyl stearate,
2-octyldodecyl erucate,
isostearyl isostearate.

The liquid fatty phase may represent from 0.5% to 90% by weight, preferably from 1% to 60% and even more preferably from 2% to 40% by weight relative to the total weight of the composition.

Hydrocarbon-based Block Copolymer

According to one embodiment of the invention, the composition contains, besides the resin, a hydrocarbon-based block copolymer also known as a block copolymer, preferably a block copolymer that is soluble in a liquid fatty phase as defined above.

The copolymer may contain at least one block with a glass transition temperature preferably of less than 20° C., preferably less than or equal to 0° C., preferably less than or equal to −20° C. and more preferably less than or equal to −40° C. The glass transition temperature of the said block may be between −150° C. and 20° C. and especially between −100° C. and 0° C.

In this case, when the resin has at least one glass transition temperature, the difference between the glass transition temperatures of the resin and of the copolymer is generally greater than 20° C., preferably greater than 40° C. and better still greater than 60° C. When the resin has at least one glass transition temperature, the block copolymer is advantageously a plasticizer for the resin described previously. The term “resin plasticizer” means a compound which, when combined in sufficient amount with the resin, lowers the glass transition temperature of the resin as defined previously. The plasticizer compound especially lowers the glass transition temperature of the polymer by at least 2, 3 or 4° C. and preferably from 5° C. to 20° C. In one preferred embodiment, the plasticizer compound especially lowers the glass transition temperature of the polymer by at least 2, 3 or 4° C. and preferably from 5° C. to 20° C.

The block copolymer may be chosen from optionally hydrogenated diblock, triblock, multiblock or radial block copolymers, and mixtures thereof.

Block copolymers comprising at least one styrene block and at least one block comprising units chosen from butadiene, ethylene, propylene, butylene and isoprene or a mixture thereof are especially preferred.

Diblock copolymers that may be mentioned include styrene/ethylene-propylene copolymers (consisting of a styrene block and of a block obtained from ethylene and propylene), styrene/ethylene-butylene copolymers, styrene/butadiene copolymers and styrene/isoprene copolymers. Such copolymers are especially sold under the name Kraton® G1701E by the company Kraton Polymers.

Triblock copolymers that may be mentioned include styrene/ethylene-propylene/styrene copolymers, styrene/ethylene-butylene/styrene copolymers, styrene/ethylene-butadiene/styrene copolymers, styrene/isoprene/styrene copolymers and styrene/butadiene/styrene copolymers. Triblock polymers are especially sold under the names Kraton® G1650, Kraton® G1652, Kraton® D1101, Kraton® D1102 and Kraton® D1160 by the company Kraton Polymers.

A mixture of a diblock copolymer and of a triblock copolymer may especially be used as block copolymer. In this embodiment, the diblock copolymer and the triblock copolymer are advantageously chosen from block copolymers comprising at least one styrene block and at least one block comprising units chosen from butadiene, ethylene, propylene, butylene and isoprene.

An example that may be mentioned is the product sold under the reference Kraton G 1657 M, which is a mixture of styrene/ethylene-butylene diblock copolymer and of styrene/ethylene-butylene/styrene triblock copolymer in 30/70 proportions, the glass transition temperature of the ethylene-butylene block being equal to about −60° C.

It is also possible to use a mixture of hydrogenated styrene/butylene-ethylene/styrene triblock copolymer and of hydrogenated ethylene/propylene/styrene star polymer. Such mixtures are sold, for example, by the company Penreco under the trade names Versagel® M5960 and Versagel® M5670.

The hydrocarbon-based block copolymer may be present in any amount including an amount of between 0.1% and 25% by weight and especially from 0.5% to 15% by weight relative to the weight of the composition.

The mass ratio between the hydrocarbon-based resin and the hydrocarbon-based block copolymer is preferably between 80/20 and 40/60 and especially between 75/25 and 50/50.

The composition may comprise an aqueous phase, which comprises water and/or at least one water-soluble solvent.

The term “water-soluble solvent” denotes in the present invention a compound that is liquid at room temperature and water-miscible (miscibility with water of greater than 50% by weight at 25° C. and atmospheric pressure).

The water-soluble solvents that may be used in the compositions according to the invention may also be volatile.

Among the water-soluble solvents that may be used in the compositions according to the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and possibly the water-miscible solvent) may be present in a content ranging from 5% to 95% by weight, preferably ranging from 10% to 80% by weight and preferentially ranging from 15% to 60% by weight relative to the total weight of the composition.

The composition according to the invention may contain emulsifying surfactants, present especially in a proportion ranging from 0.1% to 30% by weight, better still from 1% to 15% and better still from 2% to 10% by weight relative to the total weight of the composition.

The composition according to the invention may comprise at least one agent for structuring the oily phase, chosen from:
the semi-crystalline polymers described, for example, in document EP 1 396 259;
mineral lipophilic gelling agents, for instance optionally modified clays, for instance hectorites modified with a C₁₀-C₂₂ fatty-acid-ammonium chloride, for instance hectorite modified with distearyldimethylammonium chloride, for instance the product sold under the name Bentone 38V® by the company Elementis. Mention may also be made of pyrogenic silica that has optionally been hydrophobically surface-treated, the particle size of which is less than 1 μm;
molecular organo-gelling agents, especially those described in the document “Specialist Surfactants” edited by D. Robb, 1997, pp. 209-263, chapter 8 by P. Terech, in European patent applications EP-A-1 068 854 and EP-A-1 086 945 or in patent application WO-A-02/47031;
partially or totally crosslinked elastomeric organopolysiloxanes, of three-dimensional structure, such as those sold under the names KSG6®, KSG16® and KSG18® by the company Shin-Etsu;
polycondensates of polyamide type comprising at least one carboxylic acid end group esterified or amidated with at least one monoalcohol or one monoamine containing from 12 to 30 linear and saturated carbon atoms, and in particular ethylenediamine/stearyl dilinoleate copolymers such as the product sold under the name Uniclear 100 VG® by the company Arizona Chemical;
silicone polyamides of the polyorganosiloxane type, such as those described in documents U.S. Pat. No. 5,874,069, U.S. Pat. No. 5,919,441, U.S. Pat. No. 6,051,216 and U.S. Pat. No. 5,981,680, for instance those sold under the reference Dow Corning 2-8179 Gellant by the company Dow Corning;
galactomannans containing from one to six and in particular from two to four hydroxyl groups per saccharide, substituted with a saturated or unsaturated alkyl chain, for instance guar gum alkylated with C₁-C₆ and in particular C₁-C₃ alkyl chains, and mixtures thereof.

The composition according to the invention advantageously comprises at least one wax.

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for the invention may have a melting point of greater than or equal to 45° C. and in particular greater than or equal to 55° C.

The waxes that may be used in the compositions according to the invention are chosen from waxes that are solid at room temperature of animal, plant, mineral or synthetic origin, and mixtures thereof.

As illustrations of waxes that are suitable for the invention, mention may be made especially of hydrocarbon-based waxes, for instance beeswax, lanolin wax, Chinese insect waxes, rice bran wax, carnauba wax, candelilla wax, ouricurry wax, esparto grass wax, berry wax, shellac wax, Japan wax and sumach wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

Mention may also be made of silicone waxes and fluoro waxes.

The composition according to the invention may preferably comprise a content of waxes ranging from 5% to 20% by weight and may in particular contain from 7% to 15% by weight relative to the total weight of the composition.

The composition according to the invention may also comprise at least one pasty fatty substance.

The term “pasty fatty substance” means a lipophilic fatty compound comprising at a temperature of 23° C. a liquid fraction and a solid fraction. The term “pasty fatty substance” also means polyvinyl laurate.

The said pasty compound preferably has a hardness at 20° C. ranging from 0.001 to 0.5 MPa and preferably from 0.002 to 0.4 MPa.

The hardness is measured according to a method of penetration of a probe in a sample of compound and in particular using a texture analyzer (for example the TA-XT2i machine from Rheo) equipped with a stainless-steel spindle 2 mm in diameter. The hardness measurement is performed at 20° C. at the centre of five samples. The spindle is introduced into each sample at a pre-speed of 1 mm/s and then at a measuring speed of 0.1 mm/s, the penetration depth being 0.3 mm. The hardness value revealed is that of the maximum peak.

The liquid fraction of the pasty compound measured at 23° C. preferably represents 9% to 97% by weight of the compound. This liquid fraction at 23° C. preferably represents between 15% and 85% and more preferably between 40% and 85% by weight. The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., consisting of a liquid fraction and a solid fraction.

The liquid fraction of the pasty compound, measured at 32° C., preferably represents from 30% to 100% by weight of the compound, preferably from 80% to 100% and more preferably from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

Second Composition

The The product according to the invention comprises a second composition containing a medium, preferably a cosmetically acceptable medium.

The second composition is advantageously chosen such that it improves at least one cosmetic property of the first composition, when it is applied alone onto the said keratin material. The second composition may especially improve the comfort of the first composition, and especially reduce its tacky nature.

The second composition may also be chosen such that the product, once applied to the keratin materials, has satisfactory gloss properties.

The first composition and/or the second composition may be chosen such that the product, once applied to the keratin materials, has satisfactory gloss properties.

Gloss

Advantageously, the The product according to the invention may have a gloss of greater than or equal to 5, in particular greater than or equal to 10, especially greater than or equal to 15, more particularly greater than or equal to 20 and especially greater than or equal to 25, or even of about 30.

The term “gloss” denotes the gloss as may be measured by the following method, using apparatus of gonioreflectometer type, for instance the GRM-2000 machine sold by the company Micromodules.

The parameters adopted for this apparatus are as follows:
illumination angle: 120°
detection angle: 60°
start angle: 50°
end angle: 95°

A support of rectangular foam type 40×70 mm in size is made using a brick-red foam, for example a neoprene foam 3 mm thick with an adhesive face, for example a foam known under the trade reference RE40×70 EP3 sold by the company Joint Technique Lyonnais Ind.

A transparent adhesive plaster sold by the company 3M® under the trade reference Blenderm® FH 5000-55113, having a wear quality such that the application of a lipstick to this coating is similar to that produced on the lips, is attached to the face opposite the adhesive face of this support.

The foam support bearing the transparent adhesive plaster is then attached, by bonding by means of its adhesive face, to a metal plate 40×70 mm in size. The assembly consisting of the support bonded to the metal plate forms a specimen.

The operator produces a total of 5 specimens identical to the one described above.

One embodiment of the process for evaluating the gloss will now be described.

The operator places the specimen on a hotplate set at a temperature of 38.5° C., for example a hotplate of the type N81076 sold by the company Fisher Bioblock, and waits for the face of the support bearing the adhesive coating to reach a temperature of 33±1° C.

Once the support is at the desired temperature, the operator applies a film about 15 μm thick of the first composition to the adhesive coating. He leaves the first composition to dry, if it contains volatile solvents or oils. The operator then applies a film about 15 μm thick of the second composition onto the dry film of the first composition.

The cosmetic product, which is, for example, a lipstick, was stored at 24±2° C.

The action performed by the operator to deposit the film of product consists of a two-and-fro motion, so as to obtain a uniform deposit. The application of the product to the support is preferably performed so as to be as representative as possible of the real conditions of application of the product. The same test product is applied in an identical manner to the five same specimens prepared previously.

The film of product is left to dry, the specimen being placed on the hotplate, such that the support remains at 33±1° C. for 10 minutes.

The gloss of the film of product is measured for each of five specimens.

From the measured values, the mean gloss is established according to the following conventions:

$$\overline{Gloss} = \frac{1}{N} {\sum }_{i} {Gloss}_{i}$$

standard deviation:

$${σ}_{Gloss} = {\sqrt{\frac{N {\sum }_{i} {Gloss}_{i}^{2} - ( {\sum }_{i} {Gloss}_{i} )}{N ( N - 1 )}}}^{2}$$

95% confidence interval:

$$Gloss \pm 1.96 \sqrt{\frac{{σ}_{Gloss}}{N}}$$

where N denotes the number of measurements, i.e. 5 in the present case.

The cosmetically acceptable medium of the second composition preferably comprises a fatty substance that is non-volatile at room temperature and atmospheric pressure.

The term “non-volatile fatty substance” means a liquid or solid fatty substance in particular having a non-zero vapour pressure at an ambient temperature of 25° C. and an atmospheric pressure of 1 bar, of less than 0.02 mmHg (2.66 Pa) and better still less than 10⁻³ mmHg (0.13 Pa).

The fatty substance may be a hydrocarbon-based, silicone or fluoro fatty substance or may consist of a mixture thereof. The fatty substance may be a solid or a viscous oil.

The term “hydrocarbon-based” means a compound mainly comprising carbon and hydrogen atoms and in particular alkyl or alkenyl chains, for instance alkanes or alkenes, but also oils with an alkyl or alkenyl chain comprising one or more ether, ester or carboxylic acid groups.

The term “silicone” means a compound comprising silicon atoms.

The term “fluoro” fatty substance means a compound mainly comprising carbon, fluorine and hydrogen atoms.

The second composition advantageously contains a non-volatile liquid fatty phase. This fatty phase may represent 1% to 100%, preferably from 5% to 95%, better still from 20% to 80% and even better still from 40% to 80% of the total weight of the second composition.

The term “liquid fatty phase” means a phase comprising one or more fatty substances that are liquid at an ambient temperature of 25° C. and an atmospheric pressure of 1 bar.

According to one embodiment, the non-volatile fatty substance is a viscous non-volatile oil.

The term “viscous oil” means an oil whose viscosity at 25° C. is advantageously greater than or equal to 100 cSt, especially greater than or equal to 500 cSt or even greater than or equal to 1000 cSt. The viscous oil advantageously has a molecular mass of greater than or equal to 600 g/mol, for example greater than or equal to 700, or even 800, or even 900 g/mol.

The viscous oil especially has a viscosity at 25° C. of between 100 and 5 000 000 cSt, especially between 500 and 1 000 000 cSt, for example between 1000 and 700 000 cSt, for example between 10 000 and 500 000 cSt, for example equal to 350 cSt, 60 000 cSt or 300 000 cSt.

The viscous oil advantageously has a molecular mass of between 600 and 2 500 000 g/mol, for example between 700 and 2 000 000 g/mol, more for example between 1000 and 500 000 g/mol, more for example between 10 000 and 300 000 g/mol.

The dynamic viscosity at 25° C. of the viscous oil may be measured with a Mettler RM 180 rotary viscometer, taking into account the density of the oil in order to make the conversion into cSt.

The dynamic viscosity of the oil may be measured with a Mettler RM 180 viscometer. The Mettler RM 180 machine (Rheomat) may be equipped with different spindles depending on the order of magnitude of the viscosity that it is desired to measure. For a viscosity of between 0.18 and 4.02 Pa.s, the machine is equipped with a No. 3 spindle. For a viscosity of between 1 and 24 Pa.s, the machine is equipped with a No. 4 spindle, and for a viscosity of between 8 and 122 Pa.s, the machine is equipped with a No. 5 spindle. The viscosity is read on the machine in deviation units (DU). Reference is then made to charts provided with the measuring machine to obtain the corresponding value in poises, and then to convert it into stokes.

The spin speed of the spindle is 200 rpm.

Once the spindle is in rotation, at a constant set spin speed (in the present case 200 rpm), the viscosity value of the oil may vary over time. Measurements are taken at regular time intervals until they become constant. The viscosity value that has become constant over time is the value retained as being the dynamic viscosity value of the viscous oil.

The proportion of the non-volatile fatty substance in the second composition may be between 2% and 100% by weight relative to the weight of the second composition, for example from 5% to 80%, or even from 10% to 70%, or even from 20% to 40% by weight relative to the weight of the composition.

The viscous oil may be a hydrocarbon-based oil, a silicone oil or a fluoro oil.

The viscous oil may be chosen from the oils described in the paragraph concerning the liquid fatty phase of the first composition.

The viscous oil may especially be chosen from polybutenes and diisostearyl malate.

The viscous oil may be a silicone oil, for example a phenylsilicone oil, a silicone oil comprising fluorine atoms or a polydimethylsiloxane oil.

The non-volatile viscous silicone oil may be chosen from polyalkylsiloxanes, polyarylsiloxanes and polyalkylarylsiloxanes, and mixtures thereof.

The non-volatile silicone oil may be chosen from:
linear or branched non-volatile polydimethyl-siloxanes (PDMS);
polydimethylsiloxanes comprising alkyl, alkoxy or phenyl groups, which are pendent or at the end of a silicone chain, these groups containing from 2 to 24 carbon atoms;
phenylsilicones, for instance phenyl trimethi-cones, phenyl dimethicones, phenyltrimethylsiloxy-diphenylsiloxanes, diphenyl dimethicones, diphenyl-methyldiphenyltrisiloxanes and 2-phenylethyl trimethyl-siloxysilicates.

The polyalkylsiloxanes according to the invention include polydimethylsiloxanes, (polydimethylsiloxane) (methylvinylsiloxane) copolymers, poly(dimethyl-siloxane)(diphenyl)siloxanes, poly(dimethylsiloxane)-(diphenyl)(methylvinylsiloxane) copolymers, and mixtures thereof.

The fluorosilicone oil may correspond to the formula: 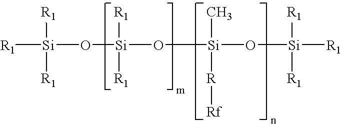
in which:
R represents a linear or branched alkylenyl group containing 1 to 6 carbon atoms, preferably a divalent methylenyl, ethylenyl, propylenyl or butylenyl group,
Rf represents a fluoroalkyl radical, especially a perfluoroalkyl radical, containing from 1 to 9 carbon atoms and preferably from 1 to 4 carbon atoms,
R₁ represents, independently of each other, a C₁-C₂₀ and preferably C₁-C₄ alkyl radical, a hydroxyl radical or a phenyl radical,
m is chosen from 0 to 150 and preferably from 20 to 100, and
n is chosen from 1 to 300 and preferably from 1 to 100.

The non-volatile fatty substance may be a solid or liquid silicone polymer.

According to one embodiment, the silicone polymer has a weight-average molecular mass of greater than or equal to 200 000 g/mol, preferably between 200 000 and 2 500 000 and preferably between 200 000 and 2 000 000 g/mol.

The viscosity of this polymer may be between 10 000 and 5 000 000 cSt, preferably between 100 000 and 1 000 000 cSt and more preferably between 300 000 and 700 000 cSt.

The silicone polymer is advantageously a non-grafted polymer, i.e. a polymer obtained by polymerization of at least one monomer, without subsequent reaction of the side chains with another chemical compound. The polymer is preferably chosen from dimethiconols, fluorosilicones and dimethicones, and mixtures thereof. The polymer is preferably a homopolymer.

In particular, the polymer used may correspond to formula (A) 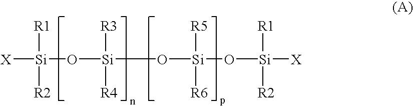
in which:
R₁, R₂, R₅ and R₆ are, together or separately, an alkyl radical containing 1 to 6 carbon atoms,
R₃ and R₄ are, together or separately, an alkyl radical containing from 1 to 6 carbon atoms or an aryl radical,
X is an alkyl radical containing from 1 to 6 carbon atoms, a hydroxyl radical, a vinyl radical, an allyl radical or an alkoxyl radical containing from 1 to 6 carbon atoms,
n and p being chosen so as to give the oil a weight-average molecular mass of greater than or equal to 600 g/mol.

The polymers of formula (A) such that R₁ to R₆ represent a methyl group and the substituent X represents a hydroxyl group are dimethiconols. Examples that are mentioned include the polymers of formula (A) such that p=0 and n is between 2000 and 40 000 and preferably between 3000 and 30 000. Mention may also be made of polymers with a molecular mass of between 1 500 000 and 2 000 000 g/mol.

According to one embodiment, the high molecular weight polymer is the dimethiconol sold by Dow Corning in a polydimethylsiloxane (5 cSt) under the reference D2-9085, the viscosity of the mixture being equal to 1550 cSt, or the dimethiconol sold by Dow Corning in a polydimethylsiloxane under the reference DC 1503. The dimethiconol (of molecular weight equal to 1 770 000 g/mol) sold by Dow Corning under the reference Q2-1403 or Q2-1401, the viscosity of the mixture being equal to 4000 cSt, is also preferred.

As silicone polymers that may be used according to the invention, mention may be made of those for which:
the substituents R₁ to R₆ and X represent a methyl group, for instance the product sold under the name SE30 by the company General Electric, and the product sold under the name AK 500 000 by the company Wacker,
the substituents R₁ to R₆ and X represent a methyl group and p and n are such that the molecular weight is 250 000 g/mol, for instance the product sold under the name Silbione 70047 V by the company Rhodia,
the substituents R₁ to R₆ represent a methyl group and the substituent X represents a hydroxyl group, for instance the products sold under the name Q2-1401 or Q2-1403 by the company Dow Corning,
the substituents R₁, R₂, R₅, R₆ and X represent a methyl group, the substituents R₃ and R₄ represent an aryl group and n and p are such that the molecular weight of the polymer is 600 000 g/mol, for instance the product sold under the name 761 by the company Rhône-Poulenc.

The silicone polymer may be introduced into the second composition in the form of a mixture with a liquid silicone, the viscosity of the said liquid silicone being between 0.5 and 10 000 cSt, preferably between 0.5 and 500 cSt and more preferably between 1 and 10 cSt.

The fluid silicone may be chosen from poly-alkylsiloxanes, polyarylsiloxanes, polyalkylaryl-siloxanes and mixtures thereof. The liquid silicone may be a volatile silicone such as a cyclic polydimethyl-siloxane comprising 3 to 7 —(CH₃)₂SiO— units.

The liquid silicone may also be a non-volatile polydimethylsiloxane silicone, especially with a viscosity of between 0.5 and 10 000 cSt and more preferably of the order of 5 cSt, for example the silicone sold under the reference DC 200 by Dow Corning.

The viscosities of the fluid silicones are measured, throughout the present patent application, according to standard ASTM D-445. The proportion of the high molecular weight polymer in the high molecular weight polymer/liquid silicone mixture is preferably between 10/90 and 20/80. The viscosity of the high molecular weight polymer/liquid silicone mixture is preferably between 1000 and 10 000 cSt.

The high molecular weight dimethicones according to the invention include the dimethicones described in patent U.S. Pat. No. 4,152,416. They are sold, for example, under the references SE30, SE33, SE54 and SE76.

The dimethicones according to the invention are, for example, compounds of formula (III) such that R₁ to R₆ and X are methyls and p=0. The molecular weight of these polymers is preferably between 200 000 and 300 000 and preferentially between 240 000 and 260 000 g/mol.

The dimethicones according to the invention include polydimethylsiloxanes, (polydimethylsiloxane) (methyl-vinylsiloxane) copolymers and poly(dimethylsiloxane)-(diphenyl)(methylvinylsiloxane) copolymers, and mixtures thereof.

The high molecular weight fluorosilicones according to the invention preferably have a molecular weight of between 200 000 and 300 000 and preferentially between 240 000 and 260 000 g/mol.

The second composition may contain a mixture of a polydimethylsiloxane with a molecular mass of between 200 000 and 300 000 g/mol and of a polydimethylsiloxane with a molecular mass of between 400 and 1000 g/mol. In this case, the mass proportion of silicone compound of high molecular weight/silicone compound of low molecular weight is preferably between 20/80 and 60/40 and preferably between 35/65 and 45/55.

The second composition may optionally contain a sparingly viscous oil, with a viscosity at 25° C. of less than 500 cSt and/or with a molecular mass of less than 600 g/mol. This oil may be one of the oils described previously in the paragraph of the liquid fatty phase of the first composition.

According to one embodiment, the second composition is transparent.

The term “transparent composition” means a transparent to translucent composition, i.e. a composition which is such that it transmits at least 40% of light and preferably at least 50% of light with a wavelength of 750 nm.

The transmission is measured using a Cary 300 Scan UV-visible spectrophotometer from the company Varian, according to the following protocol:
The composition is poured above its melting point into a spectrophotometer cuvette of square cross section with a side length of 10 mm.
The sample of the composition is then cooled for 24 hours at 35° C., and then kept in a chamber thermo-statically maintained at 20° C. for 24 hours.
The light transmitted through the sample of the composition is then measured by spectrophotometer by scanning wavelengths ranging from 700 nm to 800 nm, the measurement being performed in transmission mode.
The percentage of light transmitted through the sample of the composition at a wavelength of 750 nm is then determined.

When the second composition is transparent, it advantageously contains less than 5%, preferably less than 2% and more preferably less than 1% of pigments.

When the first or the second composition is intended to be applied to the nails, the composition preferably contains a solvent chosen from:
ketones that are liquid at room temperature, such as methyl ethyl ketone, methyl isobutyl ketone, diiso-butyl ketone, isophorone, cyclohexanone or acetone;
alcohols that are liquid at room temperature, such as ethanol, isopropanol, diacetone alcohol, 2-butoxyethanol or cyclohexanol;
glycols that are liquid at room temperature, such as ethylene glycol, propylene glycol, pentylene glycol or glycerol;
propylene glycol ethers that are liquid at room temperature, such as propylene glycol monomethyl ether, propylene glycol monomethyl ether acetate or dipropylene glycol mono-n-butyl ether;
cyclic ethers such as γ-butyrolactone;
short-chain esters (containing from 3 to 8 carbon atoms in total) such as ethyl acetate, methyl acetate, propyl acetate, isopropyl acetate, n-butyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulfoxides such as dimethyl sulfoxide;
aldehydes that are liquid at room temperature, such as benzaldehyde or acetaldehyde;
heterocyclic compounds such as tetrahydrofuran;
propylene carbonate or ethyl 3-ethoxypropionate;
mixtures thereof.

Methyl acetate, isopropyl acetate, methoxypropyl acetate, butyl lactate, acetone, methyl ethyl ketone, diacetone alcohol, γ-butyrolactone, tetrahydrofuran, propylene carbonate, ethyl 3-ethoxypropionate and dimethyl sulfoxide, and mixtures thereof, are particularly preferred.

The first and/or the second composition of the cosmetic

The product according to the invention may contain a colouring agent that may be chosen from water-soluble or liposoluble dyes, pigments and nacres, and mixtures thereof.

The term “pigments” should be understood as meaning white or coloured, mineral or organic particles, which are insoluble in the liquid organic phase and which are intended to colour and/or opacify the first composition.

The term “nacres” should be understood as meaning iridescent particles produced especially by certain molluscs in their shell, or else synthesized, which are insoluble in the medium of the first composition.

The term “dyes” should be understood as meaning generally organic compounds that are soluble in fatty substances, for instance oils, or soluble in an aqueous-alcoholic phase.

The composition according to the invention may also comprise one or more dyestuffs chosen from water-soluble dyes, and pulverulent dyestuffs, for instance the pigments, nacres and flakes that are well known to those skilled in the art. The dyestuffs may be present in the composition in a content ranging from 0.01% to 50% by weight and preferably from 0.01% to 30% by weight relative to the weight of the composition.

The solid pigments, nacres or fillers may be dispersed in the liquid fatty phase of the composition in the presence of a dispersant.

The dispersant serves to protect the dispersed particles against agglomeration or flocculation. This dispersant may be a surfactant, an oligomer, a polymer or a mixture of several thereof, bearing one or more functionalities with strong affinity for the surface of the particles to be dispersed. In particular, they can physically or chemically attach to the surface of the pigments. These dispersants also contain at least one functional group that is compatible with or soluble in the continuous medium. In particular, 12-hydroxystearic acid esters and C₈ to C₂₀ fatty acid esters of polyols such as glycerol or diglycerol are used, such as poly(12-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the composition of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The polydihydroxystearic acid and the 12-hydroxystearic acid esters are preferably intended for a hydrocarbon-based or fluorinated medium, whereas the mixtures of oxyethylene/oxypropylene dimethylsiloxane are preferably intended for a silicone medium.

The pigments may be white or coloured, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned are carbon black, pigments of D&C type, and lakes based on cochineal carmine or on barium, strontium, calcium or aluminium.

Mention may also be made of effect pigments such as particles comprising a natural or synthetic, organic or mineral substrate, for example glass, acrylic resins, polyester, polyurethane, polyethylene terephthalate, ceramics or aluminas, the said substrate possibly being coated with metallic substances, for instance aluminium, gold, silver, platinum, copper or bronze, or metal oxides, for instance titanium dioxide, iron oxide or chromium oxide, and mixtures thereof.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type, and also nacreous pigments based on bismuth oxychloride. Interference pigments, especially containing liquid crystals or multilayers, may also be used.

The water-soluble dyes are, for example, beetroot juice or methylene blue.

The composition according to the invention may comprise at least one filler, especially in a content ranging from 0.01% to 50% by weight and preferably ranging from 0.01% to 30% by weight relative to the total weight of the composition. The term “fillers” should be understood as meaning white or colourless, mineral or synthetic particles of any form, which are insoluble in the medium of the composition irrespective of the temperature at which the composition is manufactured. These fillers serve especially to modify the rheology or the texture of the composition.

The fillers may be mineral or organic of any form, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powder (Orgasol® from Atochem), poly-β-alanine powder and polyethylene powder, powders of tetrafluoroethylene polymers (Teflon®), lauroyllysine, starch, boron nitride, expanded hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

The first and/or the second composition of the invention may also contain one or more cosmetic or dermatological active agents such as those conventionally used.

As cosmetic, dermatological, hygiene or pharmaceutical active agents that may be used in the composition of the invention, mention may be made of moisturizers, vitamins, essential fatty acids, sphingolipids and sunscreens. These active agents are used in the usual amount for those skilled in the art and especially at concentrations of from 0 to 20% and in particular from 0.001% to 15% relative to the total weight of the first or second composition.

The composition may also comprise any other additive usually used in such compositions, such as water, antioxidants, fragrances, preserving agents and essential oils.

Needless to say, a person skilled in the art will take care to select this or these optional compounds, and/or the amount thereof, such that the advantageous properties of the composition are not, or are not substantially, adversely affected by the envisaged addition.

The compositions of the product may be in any form including the form of a cast product, for example in the form of a stick or wand, or in the form of a dish that may be used by direct contact or with a sponge. In particular, they find an application as cast foundations, cast makeup rouges or eyeshadows, lipsticks, lipcare bases or lipcare balms, concealer products and nail varnishes. They may also be in the form of a soft paste or a gel, a more or less fluid cream, or a liquid packaged in a tube.

The compositions of the The product according to the invention may especially constitute a cosmetic care composition for the face, the neck, the hands or the body (for example a care cream, an antisun oil or a body gel), a makeup composition (for example a makeup gel, a cream or a stick) or a composition for artificially tanning or protecting the skin.

The compositions of the The product according to the invention may be in the form of a care composition for the skin and/or the integuments or in the form of an antisun composition or a body hygiene composition, especially in the form of a deodorant. They are then especially in uncoloured form. They may then be used as a care base for the skin, the integuments or the lips (lip balms, for protecting the lips against the cold and/or sunlight and/or the wind, or care creams for the skin, the nails or the hair).

For the purposes of the invention, the term “cosmetically acceptable” means a composition of pleasant appearance, odour and feel.

Each composition of the The product according to the invention may be in any galenical form, including those normally used for topical application and especially in the form of an oily or aqueous solution, an oily or aqueous gel, an oil-in-water or water-in-oil emulsion, a multiple emulsion, a dispersion of oil in water using vesicles, the vesicles being located at the oil/water interface, or a powder. Each composition may be fluid or solid.

According to one embodiment, the first or the second composition, or both, have a continuous fatty phase preferably in anhydrous form and may contain less than 5% water and better still less than 1% water relative to the total weight of the first or second composition.

Each first and second composition may have the appearance of a lotion, a cream, an ointment, a soft paste, a salve, a solid that has been cast or moulded, especially as a stick or a dish, or a compacted solid.

Each composition may be packaged separately in the same packaging article, for example in a two-compartment pen, the base composition being delivered from one end of the pen and the top composition being delivered from the other end of the pen, each end being especially closed in a leaktight manner with a lid.

Alternatively, each of the compositions may be packaged in a different packaging article.

Preferably, the composition that is applied as the first coat is in liquid or pasty form, which is highly desirable in the case of a lipstick or an eyeliner.

The The product according to the invention may be advantageously used for making up the skin and/or the lips and/or the integuments depending on the nature of the ingredients used. In particular, the product of the invention may be in the form of a solid foundation, a lipstick wand or paste, a concealer product, an eye contour product, an eyeliner, a mascara, an eyeshadow, a body-makeup product or a skin-colouring product.

Advantageously, the second composition has care, gloss or transparency properties.

A subject of the invention is also a lip product, a varnish, a mascara, a foundation, a tattoo, a makeup rouge or an eyeshadow comprising a first and a second composition as described above.

The compositions of the product of the invention may be obtained by heating the various constituents to the melting point of the highest-melting waxes, followed by casting the molten mixture in a mould (dish or finger stall). They may also be obtained by extrusion, as described in patent application EP-A-0 667 146.

The invention is illustrated in greater detail in the following examples. The percentages are percentages by weight.

### EXAMPLE 1

First Composition: Lipstick

The lipstick below according to the invention is prepared. The proportions are in grams:

| | |
|---|---|
| Hydrogenated styrene/methylstyrene/indene | 16 |
| copolymer (Regalite R1100 from Eastman) | |
| Hydrogenated styrene/butadiene copolymer | 10 |
| (Kraton 1657 M)* | |
| Isododecane | qs 100 |
| Octyldodecanol | 3.1 |
| Pigments | 3.1 |
| Nacres | 2 |

*Kraton 1657 M is a mixture of diblock copolymer and triblock copolymer in 70/30 proportions comprising styrene blocks and ethylene-butylene blocks.

The resistance to oil and the gloss of the composition were measured according to the measuring methods indicated previously in the description.

The resistance to oil is equal to 68%. The mean gloss of a 50 micron deposit of composition, measured at 20°, is equal to 51 (±1.5). The mean gloss of a 50 micron deposit of composition, measured at 60°, is equal to 87 (±1).

Procedure:

1. A ground pigmentary mixture of the pigments is prepared in the oily phase by grinding the mixture three times in a three-roll mill.
2. The copolymer(s) and the oil are introduced into a heating pan, and the mixture is stirred using a Rayneri blender at a temperature of 100° C.
3. When a transparent liquid mixture is observed, the ground material and the nacres are introduced, and stirring of the mixture is continued on the Rayneri blender at a temperature of 100° C. for 20 minutes.
4. A quantity sufficient for 100% is made up with the oil.
5. The formulation is poured into isododecane-leaktight jars.

Second Composition: Lip Gloss

| | |
|---|---|
| Polydimethylsiloxane sold under the | 33% |
| reference AK 300 000 by Wacker (300 000 cSt) | |
| Polydimethylsiloxane sold under the | 43% |
| reference DC 200 (350 cSt) by Dow Corning | |
| Mixture of α,ω-dihydroxylated polydimethylsiloxane | 14% |
| and of polydimethylsiloxane 5 cSt | |
| sold under the reference DC 2-9085 by Dow | |
| Corning | |
| Cyclopentasiloxane | 10% |

The above written description of the invention provides a manner and process of making and using it such that any person skilled in this art is enabled to make and use the same, this enablement being provided in particular for the subject matter of the appended claims, which make up a part of the original description and including a product, such as a cosmetic product, comprising a first composition and a second composition, the first composition containing at least one resin chosen from rosin and derivatives thereof, and hydrocarbon-based resins, and mixtures thereof, the said resin having a number-average molecular mass of less than or equal to 10 000 g/mol, and the second composition, which is different from the first, comprising at least one non-volatile fatty substance.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A product comprising a first composition and a second composition which is different from the first composition, the first composition comprising at least one resin selected from the group consisting of rosin, rosin derivatives, hydrocarbon-based resins, and mixtures thereof, the resin having a number-average molecular mass of less than or equal to 10 000 g/mol, the second composition comprising at least one non-volatile fatty substance.

2. The product according to claim 1, wherein the first composition is capable of forming a film with a resistance to oil of greater than or equal to 50%.

3. The product according to claim 1, wherein the resin has a number-average molecular mass ranging from 250 to 10 000 g/mol.

4. The product according to claim 1, wherein the hydrocarbon-based resins are chosen from:
indene polymers, such as polymers derived from the polymerization in major proportion of indene monomer and in minor proportion of monomers chosen from styrene, methylindene and methylstyrene, and mixtures thereof;
indene polymers, such as pentadiene and indene resins, which are derived from the polymerization of a mixture of pentadiene and indene monomers;
pentadiene resins, such as those derived from the majority polymerization of the 1,3-pentadiene (trans or cis-piperylene) monomer and of minor monomers chosen from isoprene, butene, 2-methyl-2-butene, pentene and 1,4-pentadiene, and mixtures thereof;
cyclopentadiene dimer resins, such as those derived from the polymerization of first monomers chosen from indene and styrene, and of second monomers chosen from cyclopentadiene dimers such as dicyclopentadiene, methyldicyclopentadiene and other pentadiene dimers, and mixtures thereof;
terpenic resins derived from the polymerization of at least one monomer chosen from α-pinene, β-pinene and limonene, and mixtures thereof.

5. The product according to claim 4, comprising an indene hydrocarbon-based polymer that is a block copolymer derived from indene and from styrene or a styrene derivative.

6. The product according to claim 4, comprising an indene hydrocarbon-based polymer obtained by polymerization in major proportion of indene monomer and in minor proportion of at least one of styrene, methylindene and methylstyrene monomer, and mixtures thereof.

7. The product according to claim 1, comprising an indene hydrocarbon-based resin or polymer chosen from hydrogenated indene/methylstyrene/styrene copolymers.

8. The product according to any claim 1, wherein the resin is present in a content ranging from 0.1% to 20% by weight relative to the total weight of the composition.

9. The product according to claim 1, wherein the first composition further comprises a liquid fatty phase.

10. The product according to claim 9, wherein the liquid fatty phase of the first composition comprises at least one hydrocarbon-based volatile oil.

11. The product according to claim 9, wherein the liquid fatty phase of the first composition comprises a fatty alcohol.

12. The product according to claim 9, wherein the first composition further comprises a hydrocarbon-based block copolymer, which is soluble in the said liquid fatty phase.

13. The product according to claim 12, wherein the hydrocarbon-based block copolymer is chosen from block copolymers comprising at least one styrene block and at least one block comprising units chosen from butadiene, ethylene, propylene, butylene and isoprene, or a mixture thereof.

14. The product according to claim 13, wherein the mass ratio between the resin and the hydrocarbon-based block copolymer is between 80/20 and 40/60.

15. The product according to claim 1, wherein the first composition and/or the second composition further comprises a colouring agent chosen from liposoluble dyes, water-soluble dyes, pigments and nacres, and mixtures thereof.

16. The product according to claim 1, wherein the first and second compositions are in the form of paste.

17. The product according to claim 1, wherein the second composition is transparent.

18. The product according to claim 1, wherein the second composition contains less than 5% of pigments.

19. The product according to claim 1, wherein the product has a gloss of greater than or equal to 5.

20. The product according to claim 1, wherein the non-volatile fatty substance is a viscous oil with a viscosity at 25° C. of between 100 and 5 000 000 cSt.

21. The product according to claim 1, wherein the non-volatile fatty substance is a viscous oil with a molecular mass of between 600 and 2 500 000 g/mol.

22. The product according to claim 1, wherein the proportion of the non-volatile fatty substance in the second composition is between 2% and 100% by weight relative to the weight of the second composition.

23. The product according to claim 1, wherein the non-volatile fatty substance is chosen from hydrocarbon-based oils.

24. The product according to claim 1, wherein the non-volatile fatty substance is silicone-based.

25. The product according to claim 24, wherein the non-volatile fatty substance is chosen from polyalkylsiloxane oils, polyarylsiloxanes and polyalkylarylsiloxanes, and mixtures thereof.

26. The product according to claim 25, wherein the non-volatile fatty substance is a silicone polymer with a weight-average molecular mass of greater than or equal to 200 000 g/mol.

27. The product according to claim 26, wherein the non-volatile fatty substance is a silicone polymer with a viscosity of between 10 000 and 5 000 000 cSt.

28. The product according to claim 1, in the form of a foundation, a makeup rouge, an eyeshadow, a lipstick, a product with care properties, a mascara, an eyeliner, a nail varnish, a concealer product or a body makeup product.

29. A process for making up the skin and/or the lips and/or the integuments, which comprises applying thereto the product of claim 1, wherein:
a first coat of a first composition is applied, and
a second coat of a second composition comprising a viscous oil with a viscosity at 20° C. of greater than or equal to 100 cSt is applied at least partially over the first composition.
